(19) United States
(12) Patent Application Publication
(10) Pub. No.: US 2024/0139385 A1
(43) Pub. Date: May 2, 2024

(54) PHOTOTRIGGERABLE NITRIC OXIDE-RELEASING COMPOSITIONS AND APPLICATIONS THEREOF

(71) Applicant: University of Georgia Research Foundation, Inc., Athens, GA (US)

(72) Inventors: Hitesh HANDA, ATHENS, GA (US); Mark GARREN, ATHENS, GA (US); Elizabeth J. BRISBOIS, ATHENS, GA (US); Megan Elizabeth BROOKS, Salt Lake City, UT (US); Morgan Musialowski ASHCRAFT, Philadelphia, PA (US)

(21) Appl. No.: 18/485,737

(22) Filed: Oct. 12, 2023

Related U.S. Application Data
(60) Provisional application No. 63/379,353, filed on Oct. 13, 2022.

(51) Int. Cl. A61L 29/08; A61K 33/00; A61K 41/00; A61K 47/59; A61L 29/16; A61L 31/10; A61L 31/16

(52) U.S. Cl. CPC A61L 29/085; A61K 33/00; A61K 41/0042; A61K 47/59; A61L 29/16; A61L 31/10; A61L 31/16; A61L 2300/114

(57) ABSTRACT

Described herein are methods for controlling the release of nitric oxide from a nitric oxide releasing material. The method involves exposing the nitric oxide releasing material to light having a discrete wavelength, wherein the nitric oxide releasing material comprises a (i) a polysiloxane network and (ii) a plurality of nitric oxide-donating moieties covalently bonded to the polysiloxane network. The tunability of nitric oxide release from the nitric oxide releasing materials described herein provides unique therapeutic profiles that correspond to endogenous thresholds of nitric oxide for desired physiological response. The methods described herein can prevent bacterial growth as well as the formation of biofilms and fibrinogen on articles such as, for example, medical devices.

### CROSS-REFERENCE TO RELATED APPLICATIONS

This application claims the benefit of and priority to co-pending U.S. Provisional Patent Application No. 63/379,353, filed on Oct. 13, 2022, the contents of which are incorporated by reference herein in its entirety.

### STATEMENT REGARDING FEDERALLY SPONSORED RESEARCH OR DEVELOPMENT

This invention was made with government support under award R01HL134899 awarded by the National Institutes of Health. The government has certain rights in the invention.

### BACKGROUND

Long-term, indwelling medical devices such as vascular catheters, chest ports, stents, and pacemakers are vital for the diagnosis, mitigation, and treatment of a substantial number of diseases and ailments. However, currently available devices frequently fail due to catastrophic events commonly associated with medical device use including infection, biofouling, and device-induced thrombosis. The current standard for controlling infection is antibiotic treatment, but due to the emergence of antibiotic resistance coupled with the persistent presence of microbial biofilms, which readily form on foreign surfaces and exhibit defensive mechanisms including poor antibiotic penetration, limited nutrient uptake, and adaptive stress responses, alternative means to prevent and combat infection are needed.

Beyond the issues of infection, when medical devices are exposed to blood, proteins rapidly adsorb and a complex sequence of biochemical reactions is triggered, ultimately resulting in thrombus formation. Clots formed on the surface can totally occlude the device, obstruct device function, and can break off and move further downstream, potentially causing pulmonary embolism or myocardial infarction. Venous thromboembolism is one of the most prevalent complications associated with indwelling vascular access devices, reportedly occurring in 11-25% of critically ill patients with central venous catheters. Such complications can result in increased medical costs, extended hospitalization, or increased morbidity. To maintain device patency, clinicians currently administer anticoagulation therapies to prevent thrombosis, but systemic anticoagulation requires a careful balance between over- and under-administration to prevent clotting while avoiding hemorrhaging. For indwelling vascular access devices, heparin-based lock solutions therapies are regularly used to prevent device occlusion but can lead to complications such as low platelet counts, internal bleeding, and thrombocytopenia. Systemic anticoagulation also fails to prevent the adsorption of plasma proteins such as fibrinogen, a central player in the formation of dense fibrin networks and an anchor exploited by bacteria to increase adhesion and biofilm development. Thus, there is also a need to prevent and combat fibrinogen formation.

### SUMMARY

Described herein are methods for controlling the release of nitric oxide from a nitric oxide releasing material. The method involves exposing the nitric oxide releasing material to light having a discrete wavelength, wherein the nitric oxide releasing material comprises a (i) a polysiloxane network and (ii) a plurality of nitric oxide-donating moieties covalently bonded to the polysiloxane network. The tunability of nitric oxide release from the nitric oxide releasing materials described herein provides unique therapeutic profiles that correspond to endogenous thresholds of nitric oxide for desired physiological response. The methods described herein can prevent bacterial growth as well as the formation of biofilms and fibrinogen on articles such as, for example, medical devices.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1D show (A) the preparation of covalent SNAP-PDMS material using variable viscosity polysiloxane, aminosilane crosslinker, dibutyltin dilaurate (DBTDL) catalyst, NO donor substrate (NAPTH), and organic nitrosating agent. (B) polysiloxane viscosity determines the equivalent —OH group availability and subsequent NO donor substrate ratio to the aminosilane linker, leading to disparate performance of the NO release functionality at low and high viscosities compared to the disclosed 2550-3,570 cSt formulation (C). (D) Shows the average NO release based on the viscosity of the polysiloxane used to prepare the NO-releasing material.

FIGS. 2A-2G show the photoexcitation of SNAP-PDMS (2,550-3570 cSt) using an RGB LED with distinct diodes for (A) red, blue, and green light enables modulation of light wavelength and (B) luminal intensity based on applied voltage. NO release rates in (C) physiological conditions with protection from light (PBS 1×, 37° C.) is significantly increased in the presence of white light (D) and is tunable based on light intensity. The consistent light components of (E) red, (F) green, and (G) blue light from the diodes are also able to individually modulate NO release based on controlling the luminal intensity.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having”, “has,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” includes, but is not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. The term “alkyl” also refers to alklylene groups represented by the general formula —(CHR)ₙ—, where R is an alky group as defined above (e.g., methyl, ethyl, etc.) and n is an integer from 1 to 20. Examples of alklylene groups include, but are not limited to, methylene, ethylene, propylene, and the like.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

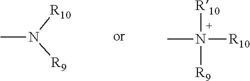

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

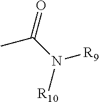

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

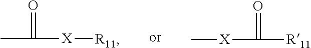

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms include, but are not limited to boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms (for example, 1-14 carbon atoms), and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “kinematic viscosity,” which is also referred to as momentum diffusivity, is defined as the ratio of the dynamic viscosity (μ) over the density of the fluid (ρ).

[symbol]

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Methods and Compositions for the Controlled Release of Nitric Oxide

Described herein are methods and compositions for controlling the release of nitric oxide from a nitric oxide releasing material. The nitric oxide releasing materials have a plurality of plurality of nitric oxide-donating moieties covalently bonded to the polysiloxane network, whereupon exposure to light having a discrete wavelength, nitric oxide is released from the polysiloxane network at a desired rate and amount. Depending upon the wavelength of the light used as well as other parameters such light intensity, the rate and amount of nitric oxide released from the nitric oxide releasing material can be fine-tuned depending upon the application or use of the nitric oxide releasing material.

The nitric oxide releasing material is exposed to light having discrete wavelength. The term “discrete wavelength” is a specified wavelength of light within a particular range of the visible light spectrum. This is distinguishable from visible light (also referred to as ambient light), which is the portion of the electromagnetic spectrum that is visible to the human eye having wavelengths from about 380 nm to about 750 nm. For example, the nitric oxide releasing material can be exposed to light having a wavelength in the range from about 400 nm to about 500 nm, from about 500 nm to about 600, or from about 600 nm to about 700 nm. Here, discrete wavelengths of light that fall within the visible light spectrum are applied to the nitric oxide releasing material.

In one aspect, the nitric oxide releasing material is exposed to light having a maximum wavelength (λₘₐₓ) in the range of about 400 nm to about 500 nm, or 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, or 500 nm, where any value can be a lower or upper endpoint of a range (e.g., 440 nm to 460 nm).

In one aspect, the nitric oxide releasing material is exposed to light having a maximum wavelength (λₘₐₓ) in the range of about 500 nm to about 600 nm, or 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, or 600 nm, where any value can be a lower or upper endpoint of a range (e.g., 505 nm to 525 nm).

In one aspect, the nitric oxide releasing material is exposed to light having a maximum wavelength (λₘₐₓ) in the range of about 600 nm to about 700 nm, or 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, or 700 nm, where any value can be a lower or upper endpoint of a range (e.g., 610 nm to 630 nm).

In another aspect, the luminous intensity of the light can be varied in addition to the wavelength of the light used. Luminous intensity is a measure of the wavelength-weighted power emitted by a light source in a particular direction and unit time per unit solid angle, based on the luminosity function. The luminal intensity of light expressed in units of lumens that is used can be varied according to voltage output to an associated light source by specification of duty cycle. Variation of the luminal intensity will affect the amount of light that falls onto the polymer surface, expressed as illuminance in unit lux. In one aspect, the duty cycle of the LED controller that is used is greater than 0% to 100%, or greater than 0%, 5%, 15%, 20%, 25%, 30%, 35%, 40%, 45%, 50%, 55%, 60%, 65%, 70%, 75%, 80%, 85%, 90%, 95%, or 100%, where any value can be a lower or upper endpoint of a range (e.g., 10% to 30%). In another aspect, luminal intensity of the light source is greater than 0 lumens to about 280,000 lumens to achieve illuminance of the NO-releasing material from 0 to 280,000 lux, or greater than 0 lumens, 25,000 lumens, 50,000 lumens, 75,000 lumens, 100,000 lumens, 125,000 lumens, 150,000 lumens, 175,000 lumens, 200,000 lumens, 225,000 lumens, 250,000 lumens, or 280,000 lumens, where any value can be a lower and upper endpoint of a range.

Depending upon the amount and duration of nitric oxide to be released, the nitric oxide releasing material can be exposed to light having a discrete wavelength for a sufficient time to release the desired amount of nitric oxide. By varying the wavelength of the light and other parameters such as the luminous intensity of the light and the amount of the nitric oxide-donating moieties present in the nitric oxide releasing material, the amount and rate of nitric oxide can be modified or fine-tuned depending upon the application.

This is contrasted with exposing the nitric oxide releasing material to visible or ambient light, where the amount of nitric oxide that is released cannot be controlled. Furthermore, the amount of nitric oxide released from the nitric oxide releasing material exposed to visible light is significantly lower when compared to the same nitric oxide releasing material exposed to light having a discrete wavelength. In one aspect, the amount of nitric oxide released from the nitric oxide material releasing material when exposed to light having a discrete wavelength is from about 2 times to about 20 times greater than the amount of nitric oxide released from the same nitric oxide material releasing material when exposed to visible light. The Examples provide evidence demonstrating that exposing the nitric oxide releasing materials to light having a discrete wavelength releases significantly more nitric oxide when compared to exposing the same materials to visible or ambient light.

In other aspects, the nitric oxide releasing material is exposed to a first light having a first discrete wavelength then subsequently exposed to a second light having a second discrete wavelength. In this aspect, different amounts and rates of nitric oxide can be released from the same nitric oxide releasing material when the material is exposed to different discrete wavelengths of light. For example, the nitric oxide releasing material can first be exposed to light having a wavelength in the range of from about 400 nm to about 500 nm for a first period of time then subsequently exposed to light having a wavelength in the range of from about 500 nm to about 600 nm for a second period of time.

The nitric oxide releasing material can be exposed to light having a discrete wavelength using equipment and techniques known in the art. In one aspect, the light is produced from diodes that produce specific or discrete wavelengths of light. In one aspect, the luminous intensity of the light can be controlled by programming of individual leads of the diode for variable duty cycle and voltage. The Examples provide non-limiting techniques for producing specific wavelengths of light and subsequently exposing the nitric oxide releasing material to the light.

In one aspect, the nitric oxide releasing material comprises (i) a polysiloxane network and (ii) a plurality of nitric oxide-donating moieties covalently bonded to the polysiloxane network.

In one aspect, the polysiloxane network in the nitric oxide releasing material is the reaction product between polysiloxane and an amine-functionalized crosslinker. In one aspect, the polysiloxane comprises one or more functional groups that can react with the amine-functionalized crosslinker. In one aspect, the polysiloxane includes two or more hydroxyl and/or amino groups. In another aspect, the polysiloxane is terminated with a hydroxyl group, which is referred to herein as a “hydroxy-terminated polysiloxane.” For example, if the polysiloxane is linear, then each end of the polysiloxane is terminated with a hydroxyl group. In other aspects, when the polysiloxane is branched, then each branch of the polysiloxane is terminated with a hydroxyl group.

In one aspect, the polysiloxane is a polydimethylsiloxane, a polydiethylsiloxane, a polydipropylsiloxane, or a polydiphenylsiloxane. In another aspect, the polysiloxane used to make the polysiloxane network is a polydimethylsiloxane, a polydiethylsiloxane, a polydipropylsiloxane, or a polydiphenylsiloxane terminated with a hydroxyl group. In one aspect, the polysiloxane has an average kinematic viscosity of about 2,500 cSt to about 4000 cSt, or about 2,500 cSt, 2,550 cSt, 2,600 cSt, 2,650 cSt, 2,700 cSt, 2,750 cSt, 2,800 cSt, 2,850 cSt, 2,900 cSt, 2,950 cSt, 3,050 cSt, 3,100 cSt, 3,150 cSt, 3,200 cSt, 3,250 cSt, 3,300 cSt, 3,350 cSt, 3,400 cSt, 3,450 cSt, 3,500 cSt, 3,550 cSt, 3,600 cSt, 3,650 cSt, 3,700 cSt, 3,750 cSt, 3,800 cSt, 3,850 cSt, 3,900 cSt, 3,950 cSt, or 4,000 cSt, where any value can be a lower and upper endpoint of range (e.g., 2,550 cSt to 3,600 cSt).

In one aspect, the amine-functionalized crosslinker includes one or more groups that react with the polysiloxane to form the polysiloxane network. In one aspect, amine-functionalized crosslinker is an amino silane compound. In one aspect, when the polysiloxane is terminated with hydroxyl groups, the hydroxyl group reacts with the silane group of the amino silane compound. In one aspect, the amino silane compound has the structure:

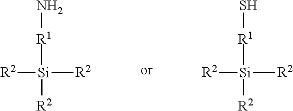

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; where each occurrence of R² is hydroxy or alkoxy. In one aspect, R¹ is a C₁-C₁₀ alkyl group such as, for example, methylene, ethylene, propylene, butylene, and the like.

A plurality of nitric oxide-donating moieties is covalently bonded to the polysiloxane network. Not wishing to be bound by theory, the polysiloxane network includes a plurality of amino groups derived from the amine-functionalized crosslinker. The amino groups can further react with additional compounds that covalently bond nitric oxide-donating moieties or precursors thereof to produce the polysiloxane network. In one aspect, a compound possessing one or more nitric oxide groups can be reacted directly with the polysiloxane network to produce the nitric oxide releasing material.

In other aspects, the polysiloxane network can be reacted with a compound that possesses one or more groups that are a precursor to the nitric oxide releasing material. In one aspect, the compound possesses one or more sulfur groups that can be subsequently nitrosylated. In one aspect, the polysiloxane network is reacted with a thiolactone. Not wishing to be bound by theory, the amino groups present in the polysiloxane network react with the thiolactone, where the thiolactone ring-opens to produce a free thiol group or ion. In one aspect. In one aspect, the thiolactone has the structure:

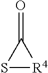

where R⁴ is a substituted or unsubstituted C₁-C₁₂ alkyl (e.g., methylene, ethylene, propylene, butylene).

In another aspect, the thiolactone has the structure:

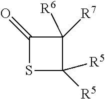

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

In one aspect, the thiolactone is N-acetylcysteine thiolactone, N-acetyl-homocysteine thiolactone, homocysteine thiolactone, butyryl-homocysteine thiolactone, or any combination thereof.

In one aspect, the nitric oxide releasing material includes a plurality of —S—NO groups. For example, when the polysiloxane network is reacted with a thiolactone as provided above, a plurality of thiol groups is produced. The thiol groups can subsequently be nitrosylated by reacting the free thiol groups with a nitrosylating agent. In one aspect, the nitrosylating agent is t-butyl nitrite, isopentyl nitrite, isobutyl nitrite, amyl nitrite, or cyclohexyl nitrite

In certain aspects, nitrosylation of the thiol group can be performed in the presence of an acid catalyst. In one aspect, the organic acid is an organic sulfonic acid having the formula RS(O)₂OH, where R is an alkyl group or aryl group as defined herein. In one aspect, R is an aryl group substituted with a C₁-C₂₀ alkyl group. In one aspect, the sulfonic acid includes, but is not limited to, dodecylbenzene sulfonic acid, dinonylnaphthalenedisulfonic acid, or 4-octylbenzenesulfonic acid. In other aspects, the organic acid can be acetic acid, formic acid, or lactic acid. In one aspect, the organic acid used is from about 0.1 weight percent to about 2 weight percent of the polysiloxane network, or about 0.1 weight percent, 0.1 weight percent, 0.2 weight percent, 0.3 weight percent, 0.4 weight percent, 0.5 weight percent, 0.6 weight percent, 0.7 weight percent, 0.8 weight percent, 0.9 weight percent, 1.0 weight percent, 1.1 weight percent, 1.2 weight percent, 1.3 weight percent, 1.4 weight percent, 1.5 weight percent, 1.6 weight percent, 1.7 weight percent, 1.8 weight percent, 1.9 weight percent, or 2.0 weight percent, where any value can be a lower and upper endpoint of range (e.g., 0.8 weight percent to 1.2 weight percent).

The nitric oxide-donating moieties covalently bonded to the polysiloxane network can vary depending upon the selection of starting materials used to produce the nitric oxide releasing material. In one aspect, the nitric oxide-donating moiety is a S-nitrosothiol. In another aspect, the S-nitrosothiol is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof. In another aspect, the nitric oxide-donating moiety is a diazeniumdiolate. In one aspect, the diazeniumdiolate is diazeniumdiolated dibutylhexanediamine or a derivative thereof.

In one aspect, the nitric oxide-donating moiety has a structure according to the following structure:

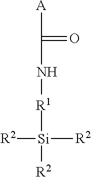

where A is a nitric oxide donor; where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; where each occurrence of R² is independently a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, a substituted or unsubstituted C₁-C₂₀ heteroalkoxy, or a bond to a polysiloxane in the plurality of polysiloxanes wherein at least two occurrences of R² are a bond to the polysiloxane network via a siloxane bond (e.g., —O—Si—O—). In one aspect, A is a S-nitrosothiol group. In another aspect, A has the structure below

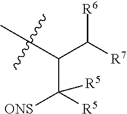

where R⁵, R⁶, and R⁷ are as defined above.

In one aspect, the nitric oxide releasing material can have the structure below:

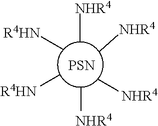

where PSN is the polysiloxane network and R⁴ is a nitric oxide-donating moiety. In one aspect, R⁴ is a S-nitrosothiol group. In another aspect, R⁴ is a residue S-nitroso-N-acetyl-penicillamine, S-nitroso-N-acetyl cysteine, S-nitroso-N-acetyl cysteamine, S-nitrosoglutathione, methyl S-nitrosothioglycolate, or a derivative thereof. In this aspect, the plurality of amino groups derived from the amine-functionalized crosslinker are covalently bonded to the nitric oxide-donating moiety.

The amount of the nitric oxide-donating moieties present in the nitric oxide releasing material can vary. In one aspect, the nitric oxide-donating moieties are present in an amount from about 0.15 micromoles per milligram of the polysiloxane network to about 0.80 micromoles per milligram of the polysiloxane network, or about 0.15 micromoles per milligram, 0.20 micromoles per milligram, 0.25 micromoles per milligram, 0.30 micromoles per milligram, 0.35 micromoles per milligram, 0.40 micromoles per milligram, 0.45 micromoles per milligram, 0.50 micromoles per milligram, 0.55 micromoles per milligram, 0.60 micromoles per milligram, 0.65 micromoles per milligram, 0.70 micromoles per milligram, 0.75 micromoles per milligram, or 0.80 micromoles per milligram, where any value can be a lower and upper endpoint of range (e.g., 0.30 micromoles per milligram to 0.70 micromoles per milligram).

In one aspect, the nitric oxide releasing material described herein can be combined with a silicone oil. Not wishing to be bound by theory, varying the amount of the silicone oil can modulate the release of nitric oxide from the nitric oxide releasing material. In one aspect, the nitric oxide releasing material and silicone oil are in an amount sufficient such that the composition has a swelling ratio of from about 0.5 to about 3, where the swelling ratio is defined by the mass of a measured portion of silicone swelled in oil divided by its original mass before swelling. In another aspect, the swelling ratio is 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0, where any value can be a lower and upper endpoint of range (e.g., 1.8 to 2.2). The viscosity of the silicone oil can also vary depending upon the application of the composition. In one aspect, the silicone oil has a viscosity of from about 10 cSt to about 500 cSt, or about 10 cSt, 25 cSt, 50 cSt, 75 cSt, 100 cSt, 125 cSt, 150 cSt, 175 cSt, 200 cSt, 225 cSt, 250 cSt, 275 cSt, 300 cSt, 350 cSt, 375 cSt, 400 cSt, 425 cSt, 450 cSt, 475 cSt, or 500 cSt, where any value can be a lower and upper endpoint of range (e.g., 25 cSt to 100 cSt).

The compositions including the silicone oil are slippery materials, which makes them useful and effective in indewelling medical devices such as, for example, catheters. The compositions described herein possess sliding angles under physiological conditions for extended periods of time. In one aspect, the sliding angles of the compositions described herein stored at 37° C. in PBS were from 15 degrees to 30 degrees over a seven day period.

Compositions composed of the nitric oxide releasing material and silicone oil can be formulated in different ways depending upon the application of the composition. In one aspect, the composition is an admixture of the nitric oxide releasing material and silicone oil. In this aspect, the nitric oxide releasing material and silicone oil are intimately mixed such that the nitric oxide releasing material is evenly (i.e., homogeneously) dispersed throughout the silicone oil. In other aspects, the nitric oxide releasing material is impregnated with the silicone oil. In one aspect, a coating of silicone oil is applied to a coating of the nitric oxide releasing material, where the silicone oil permeates (i.e., impregnates) into the nitric oxide releasing material.

By varying the relative amount of the nitric oxide releasing material and silicone oil, rate of release of the nitric oxide releasing material from the composition can be modified in addition to exposing the nitric oxide releasing material to discrete wavelengths of light as discussed herein. In certain applications, it is desirable to have sustained release of nitric oxide from the composition under physiological conditions. In one aspect, nitric oxide is released from the composition for at least 30 days, at least 45 days, at least 60 days, at least 75 days, or at least 90 days at 37° C. In another aspect, the amount of nitric oxide released from the composition is at least 0.5×10¹⁰ mol cm⁻² min⁻¹ over a period of 5 days, 10 days, 15 days, 20 days, 25 days, or 30 days. In another aspect, the amount of nitric oxide released from the composition is from about 0.5×10¹⁰ mol cm⁻² min⁻¹ to about 2.0×10¹⁰ mol cm⁻² min⁻¹ over a period of 5 days, 10 days, 15 days, 20 days, 25 days, or 30 days.

The use of biomedical devices is inevitable in hospital-based care. Unfortunately, it is also one of the leading causes of nosocomial infections. There are multiple factors that contribute to the risk of infection, including the method and duration of catheterization, quality of catheter care, and host susceptibility. The colonization of bacteria can cause infection through a number of pathways, the two most common being colonization at the insertion site by microorganisms that move through the transcutaneous part of the dermal tunnel surrounding the catheter and colonization in the intraluminal surface. Plasma proteins binding on the surface of intravascular catheters worsen this problem as they can promote bacterial adhesion. Fibrinogen is a positive acute phase protein related to blood infection, inflammatory disease, and tissue damage, ultimately causing patient mortality via trauma coagulopathy.

The compositions and methods described herein are useful in applications where it is desirable to reduce or prevent biofouling (e.g., bacterial adhesion, platelet formation, etc.) of implantable medical devices. Implantable medical devices are a leading cause of infection such as nosocomial infections. Implantable devices coated with or constructed of the compositions described herein can reduce or prevent biofouling in a subject when the device is introduced into the subject. In one aspect, the compositions described herein can reduce or prevent bacterial growth on a surface of an implantable device. In another aspect, the compositions described herein can reduce or prevent biofilm formation on a surface of an implantable device.

In another aspect, the compositions (the nitric oxide releasing material alone or in combination with a silicone oil) and methods described herein can reduce or prevent fibrinogen formation on a surface of an implantable device. Fibrinogen, a key coagulation protein, rapidly adsorbs to foreign surfaces and activates platelets. Fibrinogen contains multiple binding sites for platelet integrin α_{IIb}β₃ (GPIIbIIIa). These fibrinogen −α_{IIb}β₃ interactions play a significant role in platelet adhesion, activation, and aggregation that ultimately leads to a clot formation. To prevent thrombosis, a surface that can resist both fibrinogen binding and platelet activation is highly desirable; however, no surface reported to date is able to accomplish this.

Hemocompatibility of blood-contacting biomaterials is highly dependent on the suppression of both the contact coagulation pathway (i.e., fibrin formation) and the activation of circulating platelets. The nonthrombogenic surfaces that are currently available are designed to inhibit fibrin formation, but these surfaces do not prevent the parallel hemostatic pathway of platelet adhesion/activation and therefore are minimally effective. Thrombus and biofilm formation are highly related, where fouling of these devices is the most common clinical complication, either through protein adsorption leading to thrombus formation or bacterial adhesion resulting in infection. The compositions (the nitric oxide releasing material alone or in combination with a silicone oil) and methods described herein can prevent platelet adhesion on a surface of an article.

In one aspect, the implantable device is a urinary catheter, artificial heart valve, a vascular catheter, a graft, or a stent. In other aspects, the device is intended to contact human blood or tissue. In one aspect, the device is a hemodialysis device or a component thereof.

The compositions described herein (the nitric oxide releasing material alone or in combination with a silicone oil) can be incorporated into devices in a number of different ways. In one aspect, the devices can be coated with a composition as described herein. In one aspect, the coating composition can include an admixture of the nitric oxide releasing material and silicone oil. In another aspect, a coating of the nitric oxide releasing material can be applied to a surface of the device to produce a first coating followed by applying a coating of silicone oil on the first coating. The coating of the device can be performed using techniques known in the art such as, for example, spraying or dipping the device with the nitric oxide releasing material and silicone oil. The coating thickness can vary as well depending upon the device and application selected. In one aspect, the nitric oxide releasing material coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In other aspects, the compositions described herein (the nitric oxide releasing material alone or in combination with a silicone oil) can be used to fabricate a device. For example, when the device is composed of rubber or includes a rubber component, the rubber can be prepared such that the composition described herein is dispersed throughout the rubber to produce a nitric oxide releasing rubber. Once the nitric oxide releasing rubber has been produced, it can be used to produce devices (e.g., medical implantable devices).

### Aspects

Aspect 1. A method for controlling the release of nitric oxide from a nitric oxide releasing material, the method comprising exposing the nitric oxide releasing material to light having a discrete wavelength, wherein the nitric oxide releasing material comprises a (i) a polysiloxane network and (ii) a plurality of nitric oxide-donating moieties covalently bonded to the polysiloxane network.

Aspect 2. The method of Aspect 1, wherein the nitric oxide releasing material is exposed to light having a maximum wavelength (λₘₐₓ) in the range of about 400 nm to about 500 nm.

Aspect 3. The method of Aspect 1, wherein the nitric oxide releasing material is exposed to light having a maximum wavelength (λₘₐₓ) in the range of about 500 nm to about 600 nm.

Aspect 4. The method of Aspect 1, wherein the nitric oxide releasing material is exposed to light having a maximum wavelength (λₘₐₓ) in the range of about 600 nm to about 700 nm.

Aspect 5. The method of any one of Aspects 1 to 4, wherein the light has a luminous intensity greater than 0 lumens to about 280,000 lumens.

Aspect 6. The method of any one of Aspects 1 to 5, wherein the nitric oxide releasing material is exposed to a first light having a first discrete wavelength then subsequently exposed to a second light having a second discrete wavelength.

Aspect 7. The method of any one of Aspects 1 to 6, wherein the amount of nitric oxide released from the nitric oxide material releasing material when exposed to light having a discrete wavelength is from about 2 times to about 20 times greater than the amount of nitric oxide released from the same nitric oxide material releasing material when exposed to visible light.

Aspect 8. The method of any one of Aspects 1 to 7, wherein the nitric oxide-donating moiety comprises an S-nitrosothiol

Aspect 9. The method of any one of Aspects 1 to 7, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

Aspect 10. The method of any one of Aspects 1 to 9, wherein the polysiloxane network comprises a polysiloxane crosslinked with an amine-functionalized crosslinker.

Aspect 11. The method of Aspect 10, wherein the polysiloxane comprises a polydimethylsiloxane, a polydiethylsiloxane, a polydipropylsiloxane, or a polydiphenylsiloxane.

Aspect 12. The method of Aspect 10 or 11, wherein the polysiloxane has an average kinematic viscosity of about 2,500 cSt to about 4,000 cSt.

Aspect 13. The method of any one of Aspects 1 to 12, wherein the nitric oxide-releasing material is produced the method comprising:
crosslinking a polysiloxane with an amine-functionalized crosslinker to produce a polysiloxane network
covalently attaching a thiolactone to the polysiloxane network to produce a thiol-functionalized polysiloxane network; and
nitrosating a thiol group in the thiol-functionalized polysiloxane network in the presence of an organic acid to produce the nitric oxide-releasing material.

Aspect 14. The method of Aspect 13, wherein the organic acid comprises an organic sulfonic acid.

Aspect 15. The method of Aspect 13, wherein the organic acid comprises dodecylbenzene sulfonic acid, dinonylnaphthalenedisulfonic acid, 4-octylbenzenesulfonic acid, acetic acid, formic acid, or lactic acid.

Aspect 16. The method of Aspect 13, wherein the organic acid is dodecylbenzene sulfonic acid.

Aspect 17. The method of Aspect 13, wherein the organic acid is in the amount of from about 0.1 weight percent to about 2 weight percent of the polysiloxane network.

Aspect 18. The method of Aspect 13, wherein the polysiloxane comprises a hydroxy-terminated polysiloxane.

Aspect 19. The method of Aspect 13, wherein the amine-functionalized crosslinker has the structure

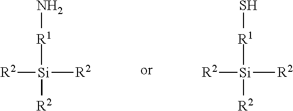

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
where each occurrence of R² is hydroxy or alkoxy.

Aspect 20. The method of Aspect 19, wherein each occurrence of R² is a hydroxy, methoxy or ethoxy.

Aspect 21. The method of Aspect 19, wherein R¹ is a substituted or unsubstituted C₁-C₁₂ alkyl or a substituted or unsubstituted C₁-C₁₂ aminoalkyl.

Aspect 22. The method of Aspect 19, wherein R¹ is methylene, ethylene, propylene, or butylene.

Aspect 23. The method of Aspect 13, wherein the thiolactone has the structure

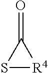

where R⁴ is a substituted or unsubstituted C₁-C₁₂ alkyl.
Aspect 24. The method of Aspect 13, wherein the thiolactone has the structure

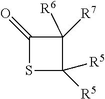

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

Aspect 25. The method of Aspect 13, wherein the thiolactone is selected from the group consisting of N-acetylcysteine thiolactone, N-acetyl-homocysteine thiolactone, homocysteine thiolactone, and butyryl-homocysteine thiolactone.

Aspect 26. The method of Aspect 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, or methyl S-nitrosothioglycolate.

Aspect 27. The method of Aspect 1, wherein the nitric oxide-donating moieties are present in an amount from about 0.15 micromoles per milligram of the polymer matrix to about 0.80 micromoles per milligram of the polysiloxane network.

Aspect 28. The method of any one of Aspects 1 to 27, wherein the nitric oxide-releasing material is present in a silicone oil.

Aspect 29. The method of Aspect 28, wherein the silicone oil has a viscosity of from about 10 cSt to about 500 cSt.

Aspect 30. The method of Aspect 28 or 29, wherein nitric oxide releasing material and silicone oil are in an amount sufficient such that the composition has a swelling ratio of from about 0.5 to about 3.

Aspect 31. The method of any one of Aspects 28 to 30, wherein the composition comprises an admixture of the nitric oxide releasing material and silicone oil.

Aspect 32. The method of any one of Aspects 28 to 30, wherein the nitric oxide releasing material is impregnated with the silicone oil.

Aspect 33. The method of any one of Aspects 1 to 32, wherein the nitric oxide releasing material is part of an article

Aspect 34. The method of Aspect 33, wherein the nitric oxide releasing material comprises a coating on at least one surface of an article.

Aspect 35. The method of Aspect 33, wherein the article comprises one or more components fabricated with the nitric oxide releasing material.

Aspect 36. The method of any one of Aspects 33 to 35, wherein the article comprises a medical device.

Aspect 37. The method of Aspect 36, wherein the device is an implantable device.

Aspect 38. The method of Aspect 36, wherein the device is selected from the group consisting of: a vascular catheter, a urinary catheter, other catheters, a coronary stent, a wound dressing, and a vascular graft.

Aspect 39. A method of preventing bacterial growth on an article, the method comprising exposing the article of any one of Aspects 33 to 38 to light having a discrete wavelength for a sufficient time to release nitric oxide from the nitric oxide releasing material.

Aspect 40. A method of preventing biofilm formation on an article, the method comprising exposing the article of any one of Aspects 33 to 38 to light having a discrete wavelength for a sufficient time to release nitric oxide from the nitric oxide releasing material.

Aspect 41. A method of preventing fibrinogen formation on an article, the method comprising exposing the article of any one of Aspects 33 to 38 to light having a discrete wavelength for a sufficient time to release nitric oxide from the nitric oxide releasing material.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Materials and Methods

Fabrication of SNAP-PDMS Material. SNAP-PDMS films were fabricated according to prior report by Hopkins et al.² OH-terminated polysiloxane is dissolved in toluene (160 mg mL⁻¹) and combined with 3-aminopropyltrimethoxy silane (APTMS, 30 mg mL⁻¹) and dibutyltin dilaurate (DBTDL, 0.24 mg/mL). The solution is stirred for 48 h. Afterwards, 3-acetamido-4,4-dimethylthietan-2-one (NAPTH) is added (30 mg/mL) and the mixture is stirred for an additional 24 h. T-butyl nitrite (TBN) cleaned with Cyclam-B (20 mM) is added to the polymer solution (3 mL cleaned TBN: 10 mL polymer solution), with the solution left to stir for an additional 30 min. Finally, the solution is casted into a square (7 cm×7 cm) Teflon mold and allowed to dry overnight. The polymer is dried an additional 24 h under vacuum to remove residual solvent. The material is stored at −20° C. until use.

Excitation of SNAP-PDMS Films via LED. SNAP-PDMS films were exposed to different intensities of visible light using 10 mm 4-pin common anode RGB LEDs (NTE30157-10) controlled through an Arduino Nano 33 IoT board. LED brightness was controlled for all three diodes: red (620-630 nm), blue (460-470 nm), and green (515-525 nm) via programming of individual leads for variable voltage. White light was developed by activating all three diodes concurrently at the same intensity. Films were placed with a path length of 1 cm from the LEDs.

Measurement of NO release. SNAP-PDMS films were placed in a custom sample chamber housing the RGB LEDs in an air and watertight compartment beneath the SNAP-PDMS films. SNAP-PDMS films were submerged in phosphate-buffered saline (PBS 1×, pH 7.4, 3 mL) at 37° C. The sample chamber was purged with a nitrogen gas sweep stream (200 mL/min) passed into a Sievers 380i Nitric Oxide Analyzer (NOA). Using a chemiluminescence detection method via the reaction of NO with ozone, the real-time instantaneous surface flux of NO from the SNAP-PDMS films was determined at different light conditions (e.g., diode on/off state and intensity setpoint).

### Results

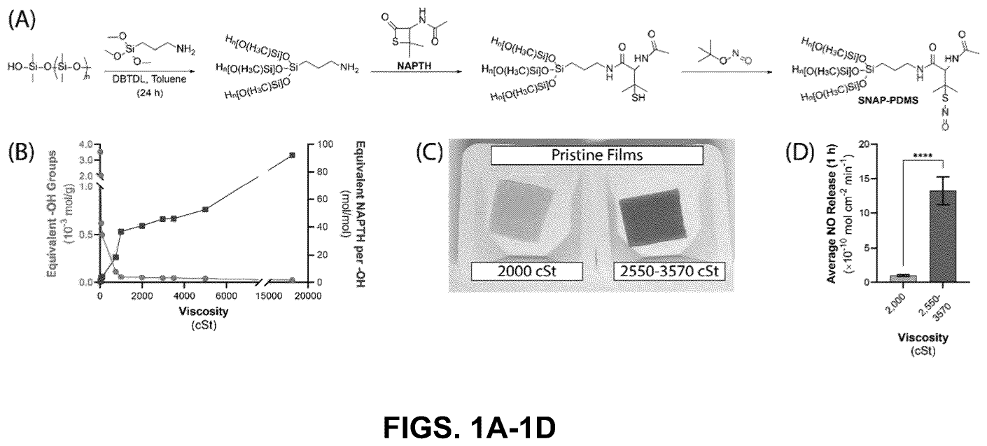

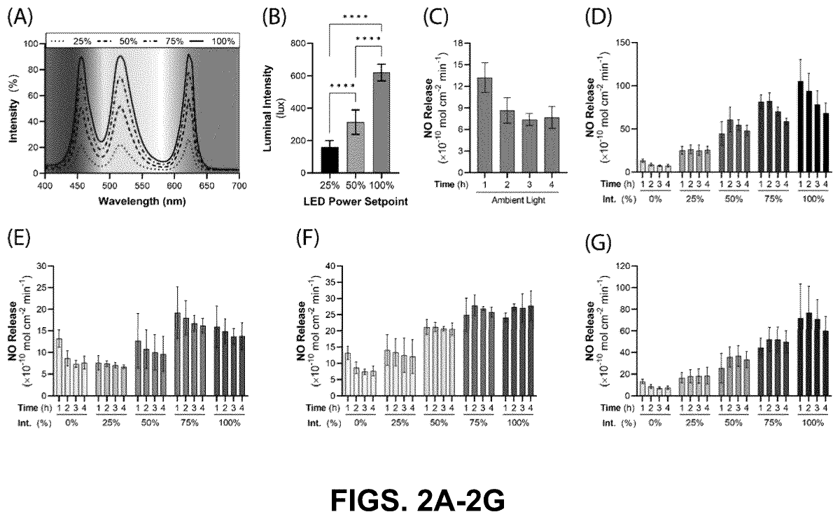

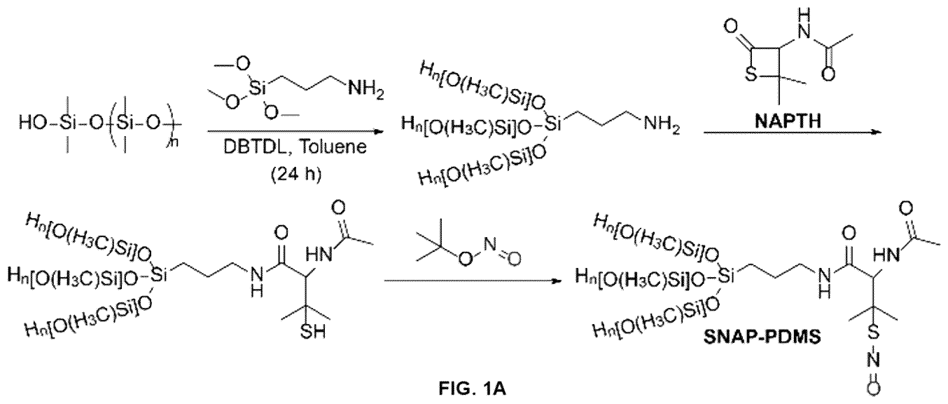

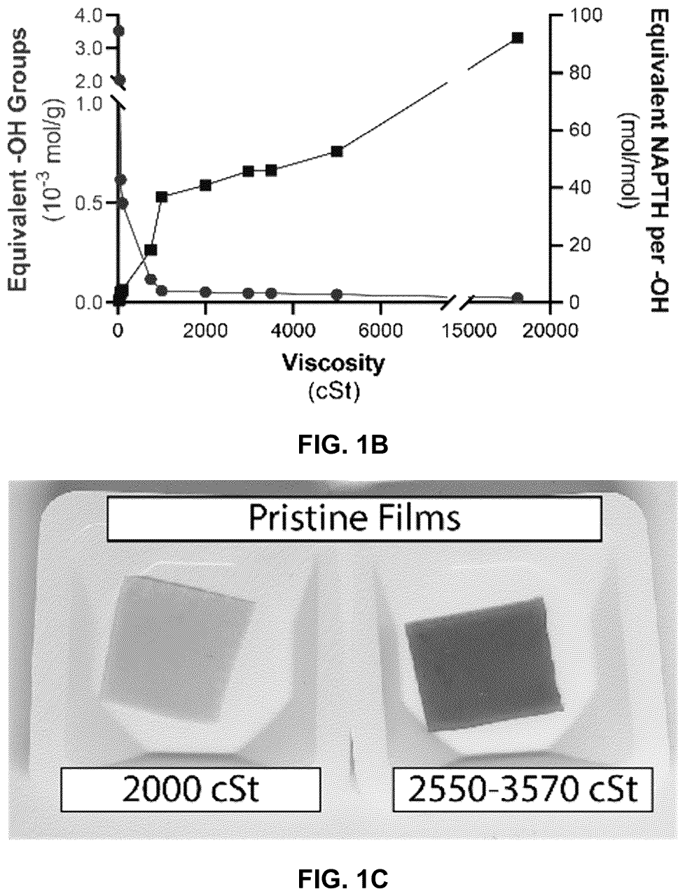

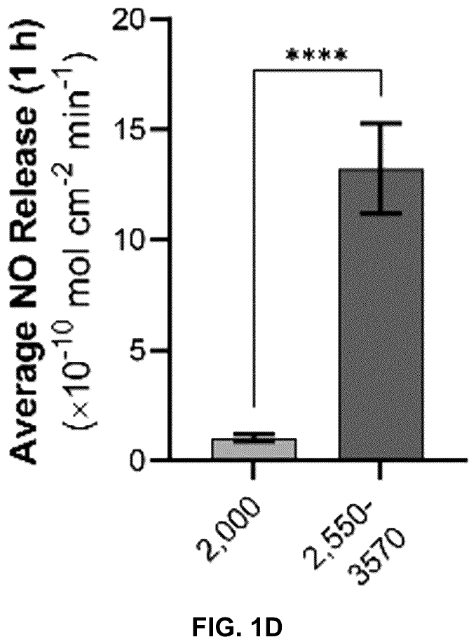

Films prepared via the covalent immobilization of nitric oxide (NO) donors to silicone rubber (SNAP-PDMS, FIG. 1A) exhibited unique NO release properties when prepared using polysiloxanes with viscosities of the range 2,550-3,570 cSt. Using prior disclosed formulations by Hopkins et. al with minor deviation (see Methods), SNAP-PDMS films were prepared with polysiloxanes of 2,000 cSt and 2,550-3570 cSt using an equimolar amount of NO donor compound (NAPTH) to aminosilane crosslinker (APTMS). In principle, lower viscosity polysiloxanes have lower molecular weights and higher equivalents of —OH groups for crosslinking (FIG. 16).¹ Different molar equivalents of the NO donor are available with respect to the —OH handles, which affects the degree of covalent integration, stability of the NO donor, and curing of PDMS films. Applying the formulation to 2,000 cSt polysiloxane, the resulting films were a lighter green color (FIG. 1C), indicating poorer integration of the NO donor. This was matched by significantly lowered NO release rates from the 2,000 cSt films (FIG. 1D) when shielded from light, corresponding to less than <8% of the steady state rate achieved with higher viscosity 2,550-3,750 counterparts. In practice, the polysiloxanes acquired in the range of 2,550-3,750 cSt had average viscosities of 2,973 cSt and molecular weight of 35,000 g mol⁻¹. This contrasts to the 2,000 cSt polysiloxanes with average molecular weight of 36,000 g mol⁻¹. Despite these minor differences in siloxane composition, significantly improved NO release rates and longevity are afforded by the 2,550-3,570 cSt (i.e., see >125 d release).²

This trend is unique and unexpected based on polysiloxane composition alone, as the modest increase in viscosity with the 2,550-3,750 cSt formulation accounts for only a ˜11% increase in molar ratio of aminosilane crosslinker to polysiloxane —OH handles. With the increased viscosity, room temperature vulcanization of the polysiloxane is facilitated in such a manner that significantly greater quantities of the NO donor functional group are preserved following nitrosation, as evident by increased green coloration associated with the S-nitrosothiol moiety (green by transmitted light) (FIG. 1C). This is associated with the ability of the 2,550-3,750 cSt composition to rapidly vulcanize following organic nitrosation, presumably due to subsequent formation of the corresponding alcohol. In effect, the increased NO donor payload achieved with the 2,550-3,750 cSt enables a stronger kinetic driving force for NO release under physiological conditions.

The baseline release rates of the 2,000 cSt films prepared were on average ˜1.02×10⁻¹⁰ mol cm⁻² min⁻¹, corresponding to a ˜400% increase in steady state release compared to the prior disclosed formulation.³ This significant increase at the same viscosity may be attributed to the increased molar ratio of NO donor to aminosilane crosslinker (˜50% higher with the 2,000 cSt formulation) and increased t-butyl nitrite content (10:1 versus 6:1 by volume with respect to the silicone mixture).

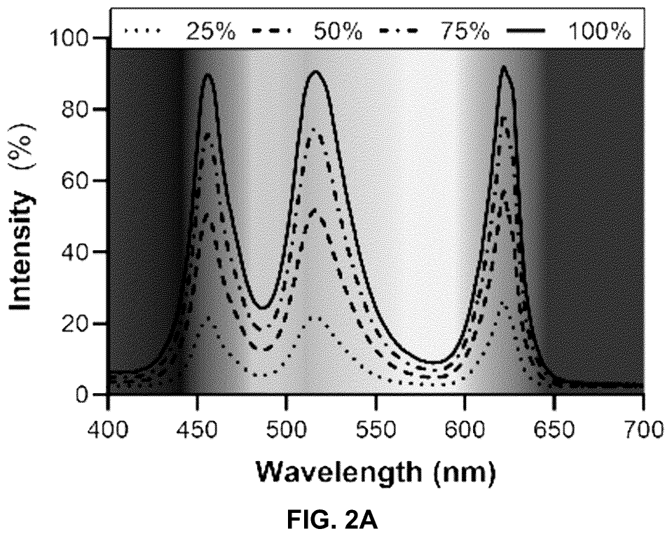

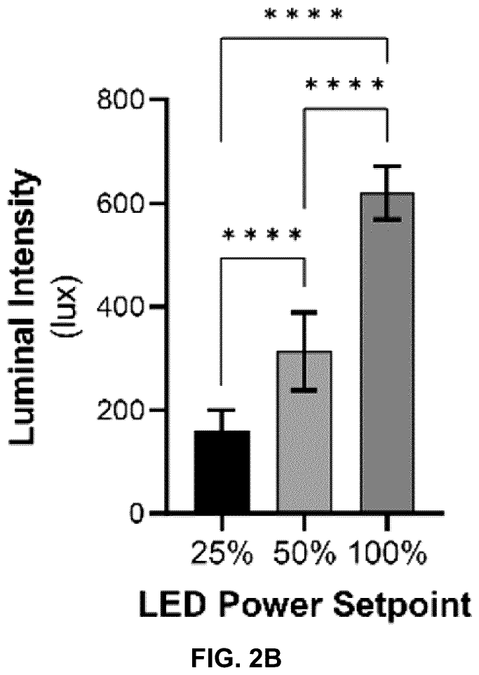

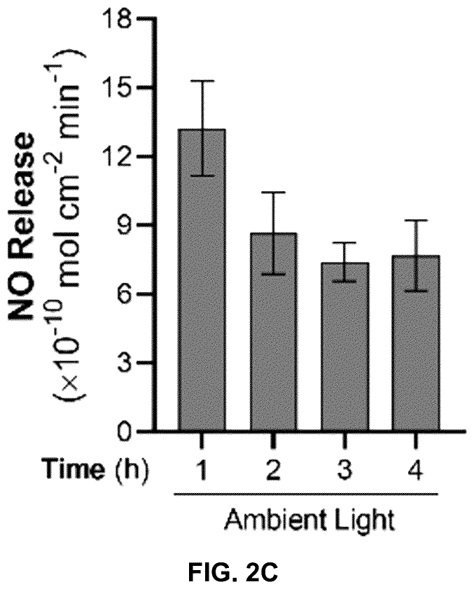

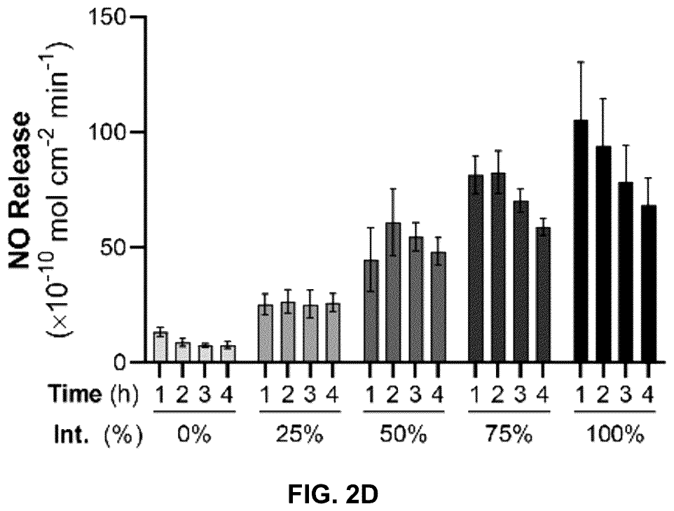

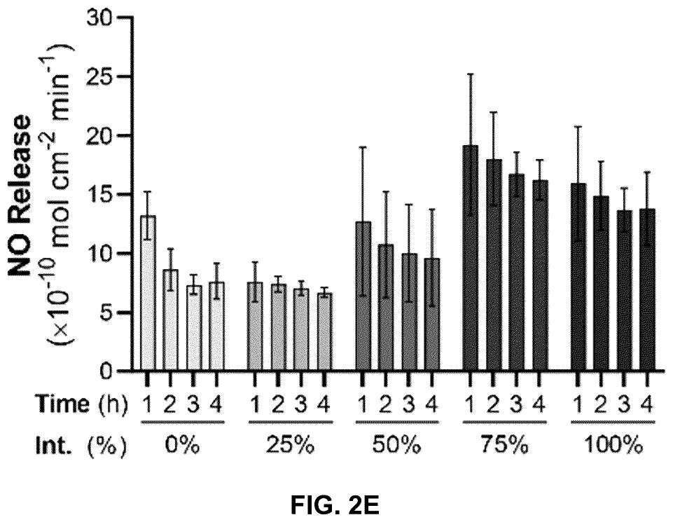

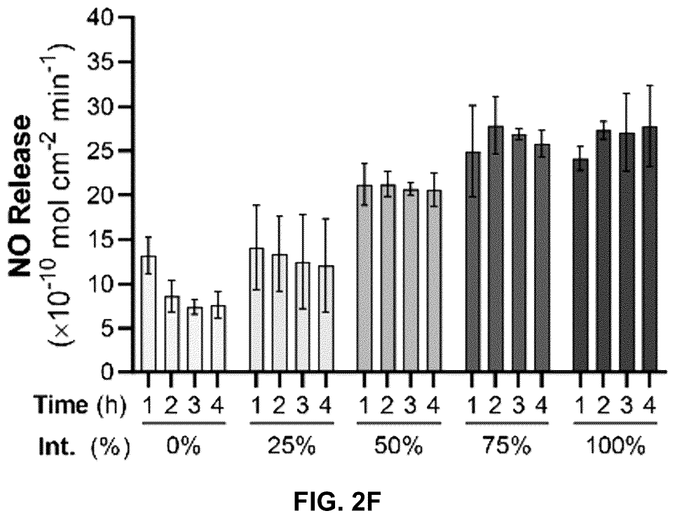

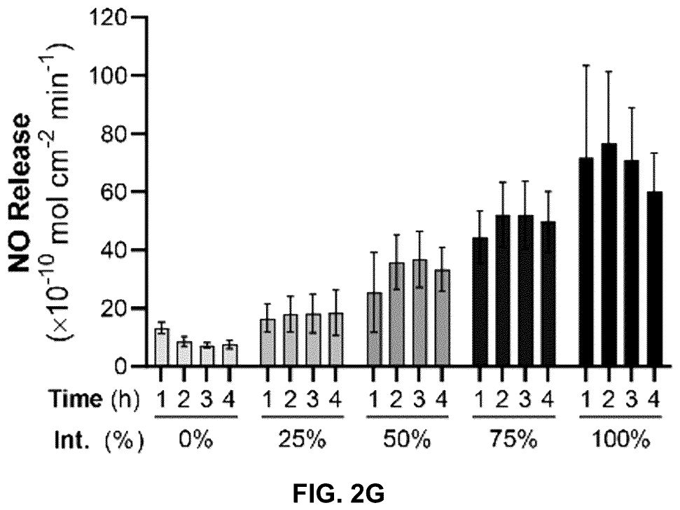

Photoexcitation of the 2,550-3570 cSt SNAP-PDMS films prepared using the herein disclosed formulation exhibited highly tunable NO release profiles based on wavelength of light and luminal intensity (FIG. 2A). Red (λₘₐₓ=621 nm), blue (λₘₐₓ=455 nm), green (λₘₐₓ=515 nm), and white (combination of all three) light were developed using 4-pin common anode LEDs. By controlling LED power setpoint, proportional changes in luminal intensity were afforded (FIG. 2B). Under protection from light, the SNAP-PDMS films exhibited NO fluxes of ˜10-15×10⁻¹⁰ mol cm⁻² min⁻¹ during 4 h studies under physiological conditions (PBS 1× at 37° C., FIG. 2C). Under white light excitation with equal intensity red, blue, and green light, the stabilized flux could be increased to ˜70-110×10⁻¹⁰ mol cm⁻² min⁻¹, corresponding to an up to ˜1,700% increase in NO release rate over dark conditions (FIG. 2D). While red light excitation leads to moderate changes in NO release rate (FIG. 2E), drastic improvements are observed with both green and blue light (FIGS. 2F-G).

Photoexcitation of the 2,550-3570 cSt SNAP-PDMS films with common RGB LEDs enables fine control of the NO release rates based on wavelength and intensity. This opens the possibility of mimicking NO release levels found endogenously to mimic different physiological responses.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1. Gelest. Silanol-Functional Silicones: Silanol-Terminated Polydimethylsiloxanes 2022. https://technical.gelest.com/brochures/reactive-silicones/silanol-functional-polymers/.
2. Hopkins, S. P.; Pant, J.; Goudie, M. J.; Schmiedt, C.; Handa, H., Achieving Long-Term Biocompatible Silicone via Covalently Immobilized S-Nitroso-N-acetylpenicillamine (SNAP) That Exhibits 4 Months of Sustained Nitric Oxide Release. ACS Appl Mater Interfaces 2018, 10 (32), 27316-27325.
3. Gierke, G. E.; Nielsen, M.; Frost, M. C., S-Nitroso-N-acetyl-D-penicillamine covalently linked to polydimethylsiloxane (SNAP-PDMS) for use as a controlled photoinitiated nitric oxide release polymer. Sci Technol Adv Mater 2011, 12 (5), 055007.

We claim:

1. A method for controlling the release of nitric oxide from a nitric oxide releasing material, the method comprising exposing the nitric oxide releasing material to light having a discrete wavelength, wherein the nitric oxide releasing material comprises a (i) a polysiloxane network and (ii) a plurality of nitric oxide-donating moieties covalently bonded to the polysiloxane network.

2. The method of claim 1, wherein the nitric oxide releasing material is exposed to light having a maximum wavelength (λₘₐₓ) in the range of about 400 nm to about 500 nm, about 500 nm to about 600 nm, or about 600 nm to about 700 nm.

3. The method of claim 1, wherein the light has a luminous intensity greater than 0 lumens to about 280,000 lumens.

4. The method of claim 1, wherein the nitric oxide releasing material is exposed to a first light having a first discrete wavelength then subsequently exposed to a second light having a second discrete wavelength.

5. The method of claim 1, wherein the amount of nitric oxide released from the nitric oxide material releasing material when exposed to light having a discrete wavelength is from about 2 times to about 20 times greater than the amount of nitric oxide released from the same nitric oxide material releasing material when exposed to visible light.

6. The method of claim 1, wherein the nitric oxide-donating moiety comprises an S-nitrosothiol

7. The method of claim 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

8. The method of claim 1, wherein the polysiloxane network comprises a polysiloxane crosslinked with an amine-functionalized crosslinker.

9. The method of claim 8, wherein the polysiloxane comprises a polydimethylsiloxane, a polydiethylsiloxane, a polydipropylsiloxane, or a polydiphenylsiloxane.

10. The method of claim 8, wherein the polysiloxane has an average kinematic viscosity of about 2,500 cSt to about 4,000 cSt.

11. The method of claim 1, wherein the nitric oxide-releasing material is present in a silicone oil.

12. The method of claim 11, wherein nitric oxide releasing material and silicone oil are in an amount sufficient such that the composition has a swelling ratio of from about 0.5 to about 3.

13. The method of claim 11, wherein the composition comprises an admixture of the nitric oxide releasing material and silicone oil.

14. The method of claim 11, wherein the nitric oxide releasing material is impregnated with the silicone oil.

15. The method of claim 1, wherein the nitric oxide releasing material is part of an article

16. The method of claim 15, wherein the nitric oxide releasing material comprises a coating on at least one surface of an article.

17. The method of claim 15, wherein the article comprises one or more components fabricated with the nitric oxide releasing material.

18. The method of claim 15, wherein the article comprises a medical device or an implantable device.

19. The method of claim 18, wherein the device is selected from the group consisting of: a vascular catheter, a urinary catheter, other catheters, a coronary stent, a wound dressing, and a vascular graft.

20. A method of preventing bacterial growth on an article, preventing biofilm formation on an article, or preventing fibrinogen formation on an article, the method comprising exposing the article of claim 15 to light having a discrete wavelength for a sufficient time to release nitric oxide from the nitric oxide releasing material.
